(19) United States
(12) Patent Application Publication
(10) Pub. No.: US 2023/0414297 A1
(43) Pub. Date: Dec. 28, 2023

(54) ULTRASOUND IMAGE PROCESSING SYSTEM AND METHOD

(71) Applicant: COSTANZIA S.R.L., Monza (IT)

(72) Inventors: Alessandro Carmelo ROTILIO, MILANO (IT); Marco SOLBIATI, MILANO (IT)

(21) Appl. No.: 18/247,733

(22) PCT Filed: Oct. 5, 2021
(86) PCT No.: PCT/IB2021/059109

§ 371 Date: Apr. 3, 2023

(30) Foreign Application Priority Data
Oct. 5, 2020 (IT) 102020000023353

(51) Int. Cl. A61B 34/20; A61B 8/08; A61B 8/00

(52) U.S. Cl. CPC A61B 34/20; A61B 8/0841; A61B 8/4254; A61B 8/463; A61B 2090/365

(57) ABSTRACT

An ultrasound image processing system including an ultrasound instrument, a medical instrument, a camera in data connection with the instruments, and an image processing device configured to process images originating from the camera and/or from the instruments to define an overall image in which the reciprocal positioning of the entire medical instrument relative to a georeferenced ultrasound image determined by the ultrasound instrument is visible.

### FIELD OF APPLICATION

The present invention relates to a system and a method for processing ultrasound images.

More in particular, the present invention to a system and a method for processing ultrasound images in interaction with a medical instrument for treating and diagnosing pathologies.

### PRIOR ART

Standard ultrasound examinations are known which have been developed to enable a medical operator to intervene with medical instruments in order to treat and diagnose pathologies, such as, for example, a needle for amniocentesis/biopsy, simultaneously with the ultrasound examination.

An operator performing an ultrasound examination positions an ultrasound probe on a patient's body in the position closest to the area in which he or she will have to intervene with the instrument, so that the ultrasound image produced on the ultrasound beam plane is displayed on a monitor, and performs the function of guiding the medical operator during the intervention.

The operator who has to intervene on the patient inserts an instrument into the body following a path deduced from the ultrasound image produced and visible on the monitor; the operator inserts the instrument into the body along the plane of the ultrasound beam, according to practice, in order to clearly visualize the needle and be able to act in an optimal manner.

A serious drawback of this approach is that when the instrument is not perfectly aligned with the ultrasound plane, the instrument is only partially visible on the monitor, and the visibility is often reduced to a single point of intersection with the ultrasound plane, making its use complex and dangerous.

The incomplete visibility of the instrument could cause an interference of the instrument itself with organs and tissues along the path of access to the organ and with the tissue that is the target for performing the diagnostic or therapeutic function for which the instrument is intended.

There is a strong need to improve the interaction between an instrument and the practice of ultrasound examinations in order to overcome the problems described, and in general to avoid interference with an internal organ/tissue not visualized/visualizable with a standard ultrasound scan.

The general object of the present invention is to provide an ultrasound image processing system/method that overcomes the problems of the prior art.

A specific object of the present invention is to provide an ultrasound image processing system/method that simplifies the interaction between the medical instrument and ultrasound images.

Another object of the present invention is to provide an ultrasound image processing system/method that is simple for a medical operator to use.

A further object of the present invention is to provide an ultrasound image processing system/method that is minimally risky for the patient.

### SUMMARY OF THE INVENTION

In a first aspect of the invention, these and other objects are achieved by an ultrasound image processing system comprising:
an ultrasound instrument, in particular adapted to contact a patient's body, coupled to first position sensors;
a medical instrument, in particular for insertion into said patient's body, coupled to third position sensors;
a camera in data connection with said instruments and adapted to capture a real scene;
an image processing device configured to process images originating from said camera and/or from said instruments;
wherein:
said ultrasound instrument is adapted to:
   perform an ultrasound scan by emitting an ultrasound beam and determining an ultrasound image, wherein said ultrasound image lies along an ultrasound beam plane;
   transmit said ultrasound image to said image processing device;
said camera is adapted to:
   pick up first position data of said ultrasound instrument by means of said first position sensors;
   transmit said first position data to said image processing device for a corresponding processing;
   acquire an image of said medical instrument;
   pick up third position data of said medical instrument by means of said third position sensors;
   transmit said image of said medical instrument and said third position data to said image processing device, for a corresponding processing;
said image processing device comprises a processing unit configured to:
   process said ultrasound image and said first position data, thereby determining a georeferenced ultrasound image;
   graphically position said georeferenced ultrasound image along said ultrasound projection beam coming out of said ultrasound instrument along said ultrasound beam plane;
   process said image of said medical instrument and said third position data, thereby determining a virtualized image of said medical instrument;
   process said virtualized image of said medical instrument and said third position data, thereby determining a virtualized and georeferenced image of said medical instrument, wherein said virtualized and georeferenced image lies along a second image plane;
   overlay said virtualized and georeferenced image of the medical instrument onto said georeferenced ultrasound image so that said second image plane intersects said ultrasound beam plane;
the overlay determining an overall image in which the reciprocal positioning of the entire medical instrument relative to said georeferenced ultrasound image is visible.

Preferably, said processing unit is further configured to:
receive, from said camera, said real scene;
graphically overlay said overall image onto said real scene received, thereby determining an augmented reality overall image;
send said augmented reality overall image to said image display;
and said image display is adapted to display said augmented reality overall image.

The ultrasound image processing system preferably comprises augmented and virtual reality glasses comprising said camera coupled to said display.

Said first position sensors are preferably provided on said ultrasound instrument. Said ultrasound instrument preferably comprises an ultrasound probe.

The ultrasound image processing system preferably comprises second position sensors provided in fixed positions relative to said ultrasound instrument.

Said third position sensors are preferably provided on said medical instrument. Said medical instrument preferably comprises an interventional needle.

Preferably, said virtualized image preferably comprises:
a first part of said medical instrument that is directly visible and directly acquirable by said camera;
a second part of the medical instrument that is not directly visible and not directly acquirable by said camera.

A positional reference system is preferably defined by said second position sensors and by respective second position data.

Said georeferenced ultrasound image and said virtualized and georeferenced image of the medical instrument are preferably georeferenced relative to said positional reference system.

In a second aspect of the invention, these and other objects are achieved by an ultrasound image processing method comprising the steps of:
providing an ultrasound instrument, in particular adapted to contact a patient's body, coupled to first position sensors;
providing a medical instrument, in particular for insertion into said patient's body, coupled to third position sensors;
providing a camera in data connection with said instruments and adapted to capture a real scene (Img_Real);
providing an image processing device configured to process images originating from said camera and/or from said instruments;
on the part of said an ultrasound instrument, carrying out the steps of:
   performing an ultrasound scan by emitting an ultrasound beam and determining an ultrasound image, wherein said ultrasound image lies along an ultrasound beam plane;
   transmitting said ultrasound image to said image processing device;
on the part of said camera carrying out the steps of:
   picking up first position data of said ultrasound instrument by means of said first position sensors;
   transmitting said first position data to said image processing device for a corresponding processing;
   acquiring an image of said medical instrument;
   picking up third position data of said medical instrument by means of said third position sensors;
   transmitting said image of said medical instrument and said third position data to said image processing device, for a corresponding processing;
on the part of said image processing device, carrying out the steps of:
   processing said ultrasound image and said first position data, thereby determining a georeferenced ultrasound image;
   graphically positioning said georeferenced ultrasound image along said ultrasound projection beam coming out of said ultrasound instrument along said ultrasound beam plane;
   processing said image of said medical instrument and said third position data, thereby determining a virtualized image of said medical instrument;
   processing said virtualized image of said medical instrument and said third position data, thereby determining a virtualized and georeferenced image of said medical instrument, wherein said virtualized and georeferenced image lies along a second image plane;
   overlaying said virtualized and georeferenced image of the medical instrument onto said georeferenced ultrasound image so that said second image plane intersects said ultrasound beam plane;
   the overlay determining an overall image wherein the reciprocal positioning of the entire medical instrument relative to said georeferenced ultrasound image is visible.

Preferably, there are provided the further steps of:
receiving, from said camera, said real scene;
graphically overlaying said overall image onto said real scene received, thereby determining an augmented reality overall image;
sending said augmented reality overall image to said image display;
and, on the part of said image display, carrying out the step of displaying said augmented reality overall image.

Preferably, there is provided a step of providing augmented and virtual reality glasses comprising said camera coupled to said display.

Preferably, there is provided a step of:
providing said first position sensors on said ultrasound instrument and
providing said third position sensors on said medical instrument.

The invention as described enables an interaction between a medical instrument and the practice of ultrasound examination such as to overcome the problems illustrated, thus achieving the following technical effects:
simplification of the interaction between the medical instrument and ultrasound images;
simplicity of use for a medical operator;
minimization of risk for the patient.

The stated technical effects/advantages and other technical effects/advantages of the invention will emerge in greater detail from the description that follows, accompanied by an example of an embodiment, given by way of illustration and not limitation with reference to the appended drawings.

### BRIEF DESCRIPTION OF THE DRAWINGS

FIG. 1 represents in general the ultrasound image processing system/method according to the invention.

FIG. 1A1 is an overall view of one embodiment of the invention with respective sensors integral with an ultrasound instrument and with a medical instrument;

FIG. 1A2 is an overall view of a variant of the embodiment of the invention with sensors applied to a patient's body;

FIG. 2 shows a detailed view of an image processing device in the system/method shown in FIG. 1;

FIG. 2A shows several components of the device in FIG. 2 in a further detail.

FIGS. 3A and 3B show details of the graphic processing performed by the device in FIGS. 2 and 2A.

FIGS. 4A and 4B respectively represent a real view (a photo) of one embodiment of the method of the invention and a corresponding schematic view.

FIG. 5 is a schematic view of the system of the invention.

### DETAILED DESCRIPTION

The invention describes a system for processing ultrasound images and tracing medical instruments.

A first aspect of the invention describes an ultrasound image processing system comprising an ultrasound instrument, a medical instrument, a camera in data connection with said instruments, and an image processing device configured to process images originating from the camera and/or from the instruments in order to define an overall image in which the reciprocal positioning of the entire medical instrument relative to a georeferenced ultrasound image determined by the ultrasound instrument is visible.

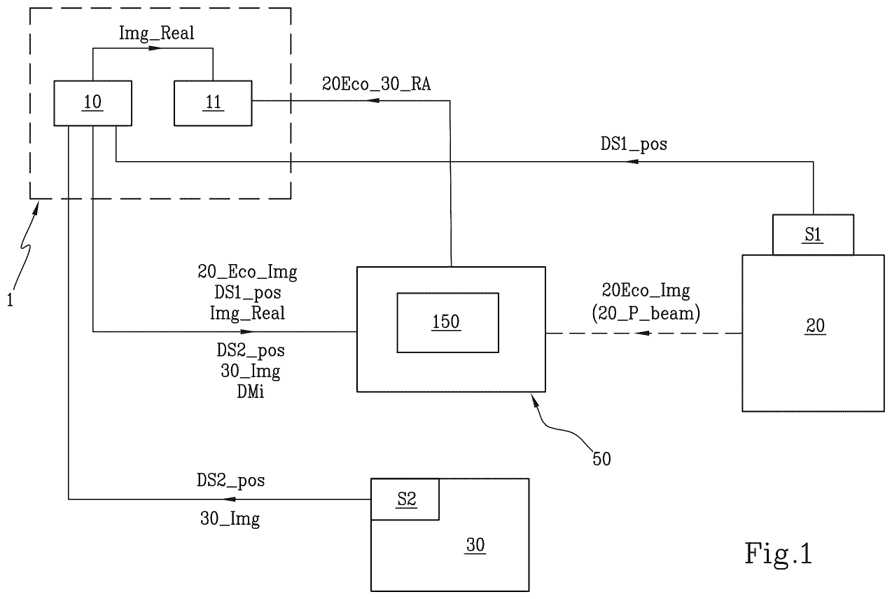

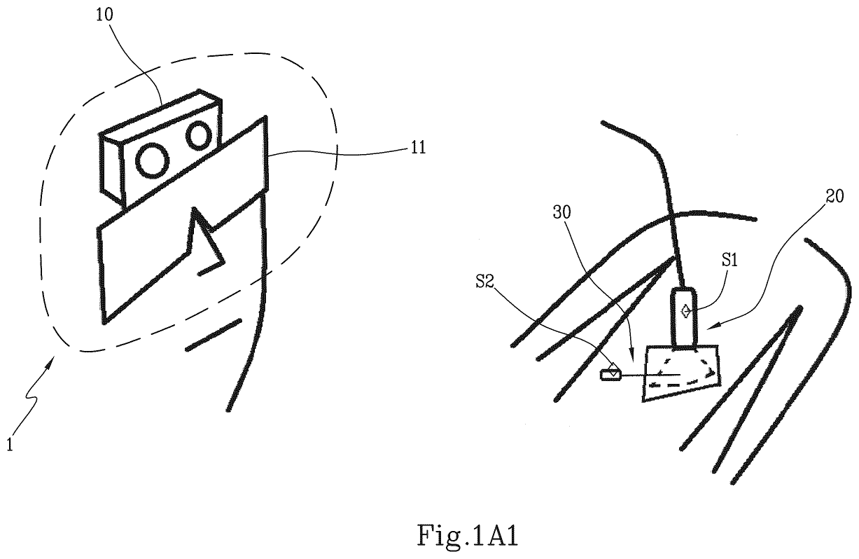

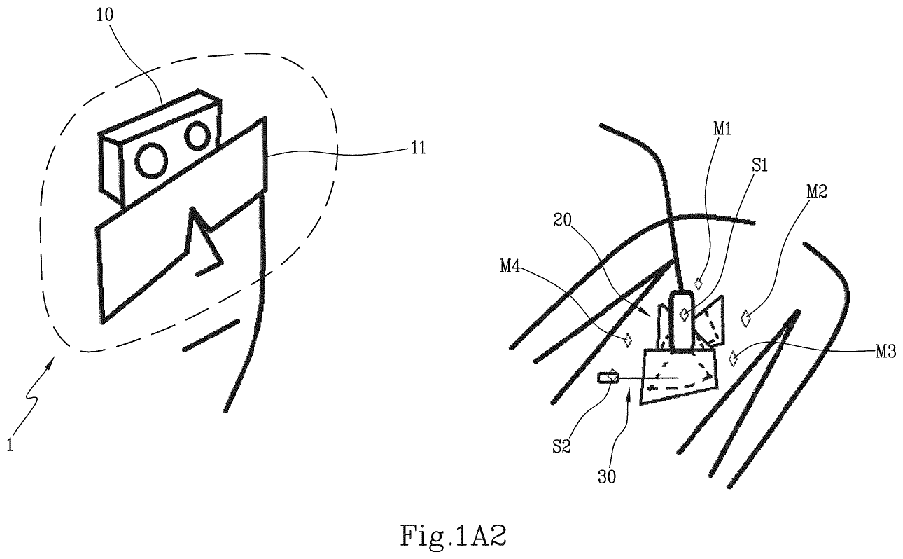

With particular reference to FIGS. 1 and 1A, the ultrasound image processing system comprises an ultrasound instrument 20, in particular adapted to contact a patient's body.

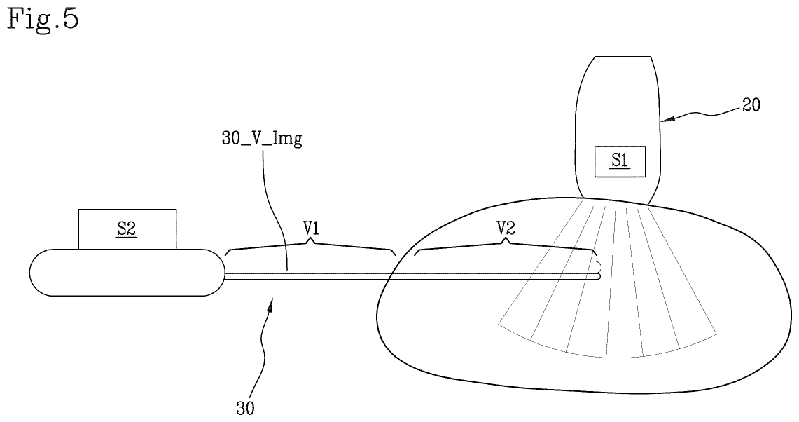

The ultrasound instrument is coupled to first position sensors S1 (FIGS. 1 and 5). According to the invention, the first position sensors S1 are provided on the ultrasound instrument 20 and, consequently, are adapted to move integrally with the ultrasound instrument itself (FIG. 1A1).

The technical effect achieved is a real-time detection of the position of the ultrasound instrument and a consequent georeferencing relative to the instrument itself.

In one variant, second position sensors (markers) Mi (i=1 . . . n) are provided, in addition to those coupled to the ultrasound instrument, and also positioned in fixed positions relative to the patient; they are shown as M1, M2, M3 and M4 in FIG. 1A2.

In other words, the second position sensors Mi are applied to the patient's body. This allows the ultrasound image to be georeferenced relative to the patient's body.

The technical effect achieved is that the ultrasound image picked up is rendered stable relative to the positioning of the second position sensors Mi applied to the patient, also with several repetitions.

Every image acquired by the ultrasound instrument thus becomes referenced and in a stable position relative to the patient's body, also independently of the successive acquisitions by the ultrasound instrument.

The stable referencing operation can be repeated multiple times to render all the stable georeferenced images visible simultaneously.

In a preferred embodiment of the invention, the ultrasound instrument 20 comprises an ultrasound probe.

With particular reference to FIG. 1, the ultrasound instrument 20 is adapted to perform an ultrasound scan by emitting an ultrasound projection beam and determining an ultrasound image 20_Eco_Img which lies along an ultrasound beam plane 20_P_beam.

The ultrasound image processing system further comprises a medical instrument 30, in particular adapted to be inserted into the patient's body.

The medical instrument 30 is coupled to third position sensors S2 (FIGS. 1 and 5). According to the invention, the third position sensors S2 are provided on said medical instrument 30 and, consequently, are adapted to move integrally with the medical instrument itself.

According to the invention, the third position sensors S2 are positioned on the medical instrument in a position proximal to a handgrip thereof, thus remaining outside the patient's body during a generation of images of interaction between the ultrasound and medical instruments.

In this manner, the third position sensors S2 can be acquired directly by a camera, as described below, as the visual path between the third sensors and the camera itself is in no way obstructed. The technical effect achieved is a real-time detection of the position of the medical instrument.

The tip of the instrument that comes into contact with and penetrates the patient's body is situated in a position distal from the handgrip.

In a preferred embodiment of the invention, the medical instrument 30 comprises an interventional needle.

In particular, the interventional needle is a needle for biopsy/amniocentesis or the like.

With reference to FIGS. 1 and 1A, the ultrasound image processing system of the invention comprises a camera 10 in data connection with the aforesaid medical and ultrasound instruments.

The camera 10, according to the invention, is adapted to capture a real scene (Img_Real) of the ultrasound instrument 20 and of the medical instrument 30 during a reciprocal interaction thereof.

In particular, according to the invention, for the purpose of detecting the reciprocal interaction of said instruments it is not necessary for there to be coplanarity between the reciprocal planes of action.

Associated with the camera 10 there is a display 11 configured to display the images/image stream acquired by the camera.

In one embodiment of the invention, shown in particular in FIG. 1A, the ultrasound image processing system comprises augmented and virtual reality glasses 1 in turn comprising the camera 10 and the display 11.

The ultrasound image processing system, according to the invention, further comprises an image processing device 50 (FIG. 1) configured to process the images Img_Real originating from the camera 10.

The image processing device 50 is further configured to process the images 20_Eco_Img and 30_Img originating respectively from the ultrasound instrument 20 and from the camera 10.

The image processing device 50 is adapted to receive the ultrasound image 20_Eco_Img from the ultrasound instrument 20.

In other words, the ultrasound instrument 20 is adapted to transmit the ultrasound image 20_Eco_Img to the image processing device 50.

According to the invention, the camera 10 is adapted to pick up first position data DS1_pos of the ultrasound instrument 20 by means of the first position sensors S1 and to transmit the first position data DS1_pos to the image processing device 50 for a corresponding processing.

Alternatively or additionally, the camera 10 is adapted to pick up second position data DM1i of the ultrasound instrument 20 by means of the second position sensors Mi and to transmit the second position data DMi to the image processing device 50 for a corresponding processing.

The camera 10 is further adapted to
acquire an image 30_Img of the medical instrument 30;
pick up third position data DS2_pos of the medical instrument 30 by means of the third position sensors S2;
transmit the image 30_Img of the medical instrument 30 and the third position data DS2_pos to the image processing device 50, for a corresponding processing.

According to the invention, the image processing device 50 comprises an electronic processing unit 150 configured to process the images originating from the camera 10 and/or from the instruments 20 and 30.

In the invention presented and in the subsequent claims, the electronic processing unit 150 is presented as divided into distinct functional modules (memory modules or operating modules) for the sole purpose of describing the functions thereof in a clear and complete manner.

The electronic processing unit 150 can consist of a single electronic device, suitably programmed to perform the functions described, and the different modules can correspond to hardware entities and/or routine software forming part of the programmed device.

Alternatively or additionally, said functions can be carried out by a plurality of electronic devices over which the aforesaid functional modules can be distributed. The electronic processing unit 150 can further rely on one or more processors to execute the instructions contained in the memory modules.

The aforesaid functional modules can also be distributed over various local or remote computers based on the architecture of the network they reside in.

The system of the invention enables numerous advantages/technical effects to be obtained for the different individuals involved.

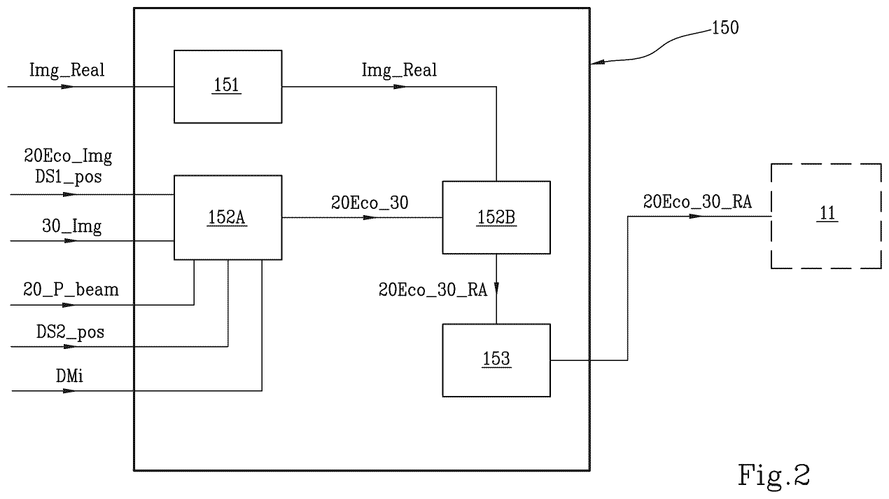

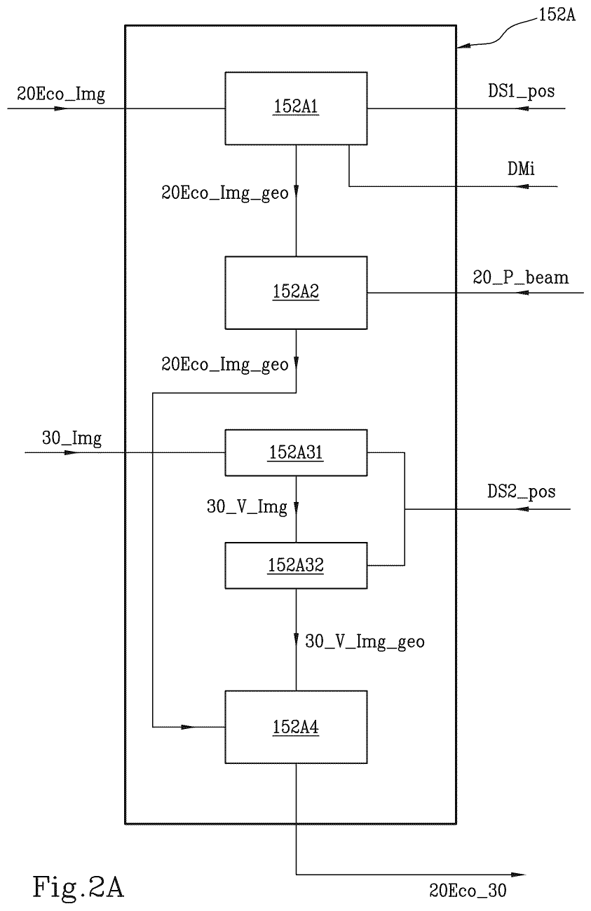

According to the invention, with particular reference to FIG. 2A, the processing unit 150 is configured to process 152A1 the ultrasound image 20_Eco_Img and the first position data DS1_pos, thereby determining a georeferenced ultrasound image 20_Eco_Img_geo.

In other words, the processing unit 150 comprises a first processing module 152A1 configured to process the ultrasound image 20_Eco_Img and the first position data DS1_pos, thereby determining a georeferenced ultrasound image 20_Eco_Img_geo.

Preferably, the ultrasound image 20_Eco_Img_geo is georeferenced relative to a positional reference system Geo_Ref defined by the second position sensors Mi and respective second position data DMi together.

The second position sensors Mi are preferably optical or electromagnetic sensors.

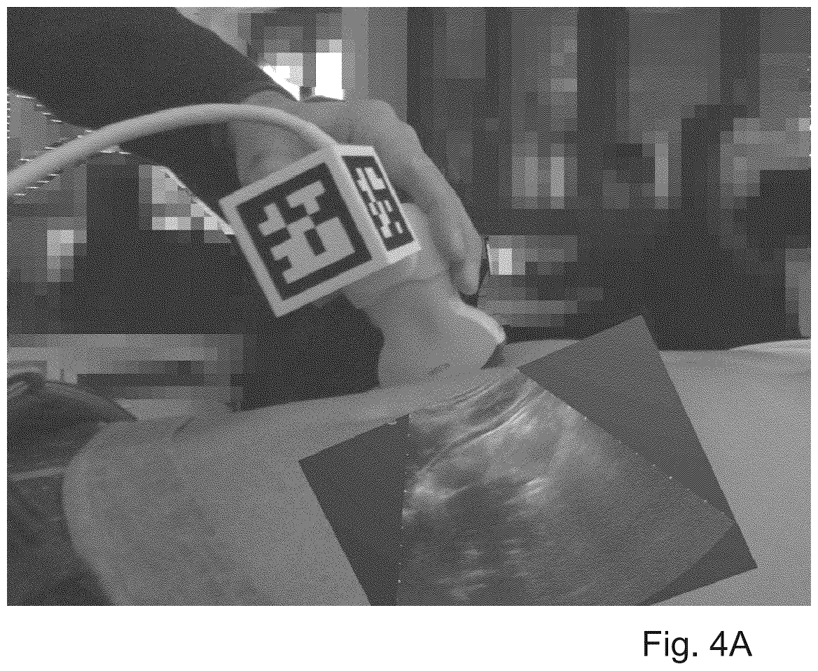

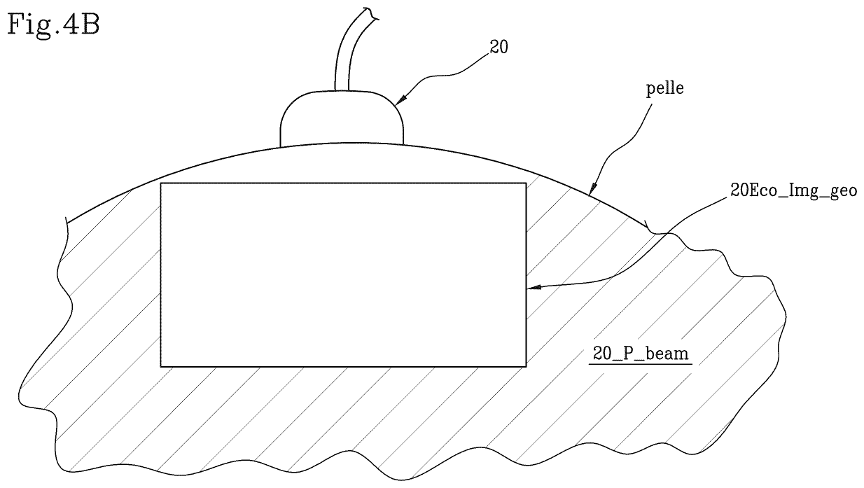

With particular reference to FIG. 4B, the processing unit 150 is further configured to graphically position 152A2 the georeferenced ultrasound image 20_Eco_Img_geo along the ultrasound projection beam coming out of the ultrasound instrument 20 along the ultrasound beam plane 20_P_beam, with the optical effect of displaying the georeferenced image 20_Eco_img_geo.

In other words, the processing unit 150 comprises a graphic positioning module 152A2 configured to graphically position the georeferenced ultrasound image 20_Eco_Img_geo along the ultrasound projection beam coming out of the ultrasound instrument 20 along the ultrasound beam plane 20_P_beam, with the optical effect of displaying the georeferenced image 20_Eco_img_geo.

FIG. 4a, by contrast, is a photo that provides a real view of the interaction between the ultrasound instrument, the emitted beam and the patient on whom the instrument is used.

Since the first position sensors S1 are positioned so as to remain outside the patient's body, in order to avoid invading the patient's body in any way with said sensors it is necessary to determine a correct theoretical position of the medical instrument inside the body also when it is not visible from the outside.

With particular reference to FIG. 4B, the processing unit 150 is further configured to process 152A31 the image of the medical instrument 30_Img and the third position data DS2_pos, thereby determining a virtualized image 30_V_Img (FIG. 2A) of the medical instrument 30.

In other words, the processing unit 150 comprises a processing module 152A31 configured to process 152A31 the image of the medical instrument 30_Img and the third position data DS2_pos, thereby determining a virtualized image 30_V_Img of the medical instrument 30.

In other words, with particular reference to FIG. 5, the virtualized image 30_V_Img comprises:
a first part V1 of the medical instrument 30 that is directly visible and directly acquirable by the camera 10, i.e. without obstacles; the first part V1 is for example a part of a needle outside the patient's body;
a second part V2 of the medical instrument 30 that is not directly visible and not directly acquirable by the camera 10, for example, an extension of the needle inside the body, not visible from outside the patient's body.

According to the invention, therefore, the processing module 152A31 is configured to graphically construct the virtualized image 30_V_Img on the basis of the position data picked up by the first position sensors S1 and a predefined conformation conf_30 of the medical instrument 30.

It follows that, in the case of a medical needle provided with first position sensors S1 and having a predefined shape conf_30, a virtualized image 30_V_Img of the needle both inside and outside the body is exactly reconstructed, ensuring that the operator can see the position of the needle in the body and the continuation of the image of the needle outside the body as if the body were open and the needle accessible.

The technical effect achieved is, therefore, that of rendering the position of the needle detectable and evaluable also in areas where this is not physically possible.

The processing unit 150 is further configured to process 152A32 the virtualized image 30_V_Img and the third position data DS2_pos, thereby determining a virtualized and georeferenced image 30_V_Img_geo of the medical instrument 30, wherein the virtualized and georeferenced image 30_V_Img_geo lies along a second image plane P_30_Img.

It may be understood that the second image plane P_30_Img is identified in terms of position in space, as it is the plane in which the virtualized and georeferenced image 30_V_Img_geo lies, in turn identified in terms of position in space by means of the virtualized image 30_V_Img—wherein said virtualized image 30_V_Img is graphically constructed on the basis of the position data picked up by the first position sensors S1 and a predefined conformation conf_30 of the medical instrument 30—and by means of the third position data DS2_pos (picked up by means of the third position sensors S2).

In particular, the virtualized image 30_V_Img_geo of the medical instrument 30 is georeferenced in reference to the positional reference system Geo_Ref.

In other words, the processing unit 150 comprises a second processing module 152A32 (FIG. 2A) configured to process the virtualized image 30_V_Img and the third position data DS2_pos, thereby determining a virtualized and georeferenced image 30_V_Img_geo of the medical instrument, in particular in reference to the positional reference system Geo_Ref, wherein the virtualized and georeferenced image 30_V_Img_geo lies along the second image plane P_30_Img.

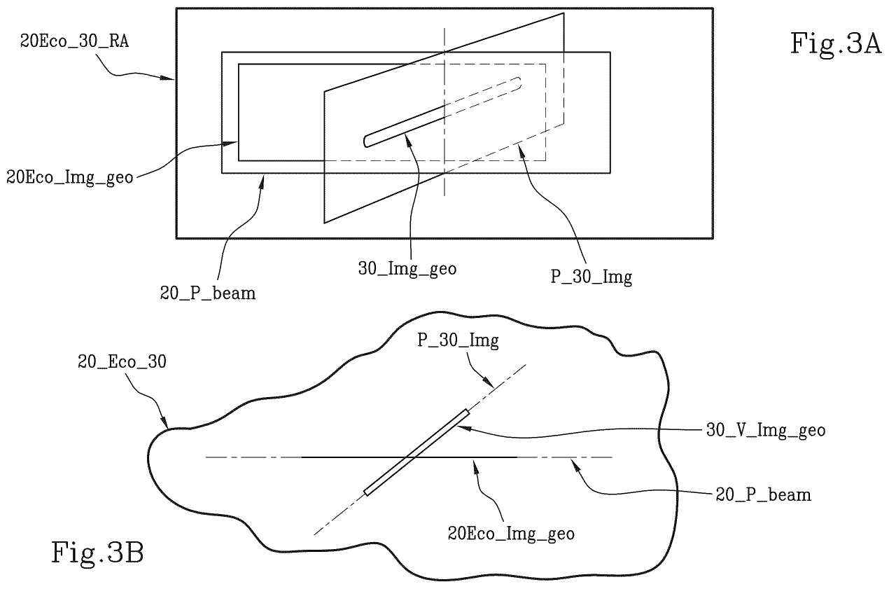

With particular reference to FIGS. 3B and 2A, the processing unit 150 is further configured to overlay 152A4 the virtualized and georeferenced image of the medical instrument 30_V_Img_geo onto the georeferenced ultrasound image 20_Eco_Img_geo so that the second image plane P_30_Img intersects the ultrasound beam plane 20_P_beam.

It may be understood that the second image plane P_30_Img intersects the ultrasound beam plane 20_P_beam, since the virtualized and georeferenced image of the medical instrument 30_V_Img_geo is overlaid onto the georeferenced ultrasound image 20_Eco_Img_geo, wherein the virtualized and georeferenced image of the medical instrument 30_V_Img_geo is defined in terms of spatial coordinates by means of the virtualized image 30_V_Img and the third position data DS2_pos, and the georeferenced ultrasound image 20_Eco_Img_geo is defined in terms of spatial coordinates by means of the ultrasound image 20_Eco_Img and the first position data DS1_pos; consequently, the intersection between the second image plane P_30_Img and the ultrasound beam plane 20_P_beam is defined in terms of spatial coordinates. In other words, the electronic processing unit 150 comprises an overlay module 152A4 configured to overlay the virtualized and georeferenced image of the medical instrument 30_V_Img_geo onto the georeferenced ultrasound image 20_Eco_Img_geo so that the second image plane P_30_Img intersects the ultrasound beam plane 20_P_beam.

The technical effect achieved by said overlay is a determination of an overall image 20Eco_30 (FIG. 3B,2a) in which the reciprocal positioning of the entire medical instrument 30 relative to the georeferenced ultrasound image 20_Eco_Img_geo is visible.

The major technical effect of this solution is that the operator always has at his or her disposal the reciprocal positions of the entire medical instrument and of the ultrasound plane and does not need to decide how to move the medical instrument to render it visible inside the body by means of an ultrasound image, as is the case in the prior art, based only on his or her own experience and in the absence of objective references. The operator can thus act upon the medical instrument with a full view of where it is positioned and also in reference to the georeferenced ultrasound image 20_Eco_Img_geo.

According to the invention, in fact, the needle is entirely visible and virtualized, i.e. both the part thereof outside the patient's body and the one inside the patient's body are visible.

With particular reference to FIG. 2A, the processing unit 150 is further configured to receive 151, from the camera 10, the real captured scene Img_Real.

In other words, the processing unit 150 comprises a receiving module 151 configured to receive the real captured scene Img_Real from the camera 10. With particular reference to FIG. 2, the processing unit 150 is further configured to graphically overlay 152B the overall image 20Eco_30 onto the real scene Img_Real received, thereby determining an augmented reality overall image 20Eco_30_RA (FIG. 3A).

In other words, the processing unit 150 comprises an overlay module 152B configured to graphically overlay the overall image 20Eco_30 onto the real scene Img_Real received, thereby determining the augmented reality overall image 20Eco_30_RA.

Furthermore, with reference to FIG. 2, the processing unit 150 is further configured to send 153 the augmented reality overall image 20Eco_30_RA (FIG. 3A) to the image display 11.

In other words, the processing unit 150 comprises a sending module 152B configured to send the augmented reality overall image 20Eco_30_RA to the image display 11.

The image display 11 is adapted to display the augmented reality overall image 20Eco_30_RA.

The technical effect achieved is the possibility of seeing, with the glasses 1 or the monitor 10, both the first sensors S1 and the third sensors S2, and of reading the interaction between the ultrasound plane and the medical instrument.

Consequently, when the medical instrument, in particular the needle, disappears into the patient's body, the reciprocal positioning of the needle and of the ultrasound plane is visible all the same.

A second aspect of the invention relates to a method for processing ultrasound images, comprising the steps of:
providing an ultrasound instrument 20, in particular adapted to contact a patient's body, coupled to first position sensors S1;
providing a medical instrument 30, in particular for insertion into the patient's body, coupled to third position sensors S2;
providing a camera (10) in data connection with said instruments (20,30) and adapted to capture a real scene Img_Real;
providing an image processing device 50 configured to process images (Img_Real; 20_Eco_Img; 30_Img) originating from the camera 10 and/or from the instruments 20,30;
on the part of said an ultrasound instrument (20), carrying out the steps of:
   performing an ultrasound scan by emitting an ultrasound projection beam and determining an ultrasound image 20_Eco_Img, wherein said ultrasound image lies along an ultrasound beam plane 20_P_beam;
   transmitting the ultrasound image 20_Eco_Img to the image processing device 50;
on the part of said camera (10), carrying out the steps of:
   picking up first position data DS1_pos of the ultrasound instrument 20 by means of the first position sensors S1;
   transmitting the first position data DS1_pos to the image processing device 50 for a corresponding processing;
   acquiring an image 30_Img of the medical instrument 30;
   picking up third position data DS2_pos of the medical instrument 30 by means of the third position sensors S2;
   transmitting the image 30_Img of the medical instrument and the third position data DS2_pos to the image processing device 50, for a corresponding processing;
on the part of the image processing device 50, carrying out the steps of:
   processing (152A1) the ultrasound image 20_Eco_Img and the first position data DS1_pos, thereby determining a georeferenced ultrasound image 20_Eco_Img_geo, in particular in reference to a positional reference system Geo_Ref;
   graphically positioning 152A2 the georeferenced ultrasound image 20_Eco_Img_geo along the ultrasound projection beam coming out of the ultrasound instrument 20 along the ultrasound beam plane 20_P_beam;
   processing 152A31 the image of the medical instrument 30_Img and the third position data DS2_pos, thereby determining a virtualized image 30_V_Img of the medical instrument;
   processing (152A32) the image of the medical instrument 30_Img and the third position data DS2_pos thereby determining a virtualized and georeferenced image (30_V_Img_geo) of the medical instrument, in reference to the positional reference system Geo_Ref, wherein the virtualized and georeferenced image 30_V_Img_geo lies along a second image plane (P_30_Img);
   overlaying (152A4) the virtualized and georeferenced image of the medical instrument 30_V_Img_geo onto the georeferenced ultrasound image 20_Eco_Img_geo so that the second image plane P_30_Img intersects the ultrasound beam plane 20_P_beam;
   the overlay determining an overall image (20Eco_30) in which the reciprocal positioning of the entire medical instrument (30) relative to the georeferenced ultrasound image 20_Eco_Img_geo is visible.

Preferably, the method comprises the further steps, on the part of the image processing device 50, of:
receiving 151, from the camera 10, the real scene Img_Real;
graphically overlaying 152B the overall image 20Eco_30 onto the real scene Img_Real received, thereby determining an augmented reality overall image 20Eco_30_RA;
sending 153 the augmented reality overall image 20Eco_30_RA to the image display 11;
and, on the part of the image display 11, carrying out the step of displaying the augmented reality overall image 20Eco_30_RA.

The method further comprises the step of providing augmented and virtual reality glasses 1 comprising the camera 10 coupled to the display 11.

Further steps of the method correspond to the previously described functionalities of the ultrasound instrument and/or medical instrument and/or camera and/or image processing device.

It may be understood from the description that, in the system and in the method of the invention, the second image plane P_30_Img corresponds to the plane in which the virtualized and georeferenced image 30_V_Img_geo lies, thus being identified in terms of position in space.

It may be understood from the description that, in the system and in the method of the invention, the virtualized and georeferenced image 30_V_Img_geo is identified in terms of position in space by means of the virtualized image 30_V_Img and the third position data DS2_pos.

It may be understood from the description that, in the system and in the method of the invention, the virtualized image 30_V_Img is graphically constructed on the basis of the position data picked up by the first position sensors S1 and a predefined conformation conf_30 of the medical instrument 30.

It may be understood from the description that, in the system and in the method of the invention, the georeferenced ultrasound image 20_Eco_Img_geo is defined in terms of spatial coordinates by means of the ultrasound image 20_Eco_Img and the first position data DS1_pos.

It may be understood from the description that, in the system and in the method of the invention, the overlay between the virtualized and georeferenced images of the medical instrument 30_V_Img_geo and the georeferenced ultrasound image 20_Eco_Img_geo determines the intersection between the second image plane P_30_Img and the ultrasound beam plane 20_P_beam, defining the intersection in terms of spatial coordinates.

An inventive system and method for processing ultrasound images have been described.

The invention as described enables an innovative interaction between a medical instrument and the practice of ultrasound examination, achieving the following technical effects:
simplification of the interaction between the medical instrument and the ultrasound images produced;
simplicity of use for a medical operator;
minimization of risk for the patient.

1.-20. (canceled)

21. An ultrasound image processing system comprising:
an ultrasound instrument, configured to contact a patient's body, coupled to first position sensors;
a medical instrument, configured for insertion into said patient's body, coupled to third position sensors;
a camera in data connection with said ultrasound instrument and medical instrument, the camera configured to capture a real scene; and
an image processing device comprising a processing unit configured to process images originating from said camera and/or from said ultrasound instrument and medical instrument; wherein:
i) said ultrasound instrument s configured to:
perform an ultrasound scan by emitting an ultrasound beam and determining an ultrasound image, said ultrasound image lying along an ultrasound beam plane; and
transmit said ultrasound image to said image processing device;
ii) said camera is configured to:
pick up first position data of said ultrasound instrument through said first position sensors;
transmit said first position data to said image processing device for a corresponding processing;
acquire an image of said medical instrument;
pick up third position data of said medical instrument through said third position sensors;
transmit said image of said medical instrument and said third position data to said image processing device, for a corresponding processing;
iii) said processing unit is configured to:
process said ultrasound image and said first position to determine a georeferenced ultrasound image;
graphically position said georeferenced ultrasound image along said ultrasound projection beam coming out of said ultrasound instrument along said ultrasound beam plane;
process said image of said medical instrument and said third position data to determine a virtualized image of said medical instrument;
process said virtualized image of said medical instrument and said third position data to determine a virtualized and georeferenced image of said medical instrument, wherein said virtualized and georeferenced image lies along a second image plane; and
overlay said virtualized and georeferenced image of the medical instrument onto said georeferenced ultrasound image so that said second image plane intersects said ultrasound beam plane, the overlay determining an overall image in which a reciprocal positioning of the entire medical instrument relative to said georeferenced ultrasound image is visible.

22. The image processing system according to claim 21, wherein
iv) said processing unit is further configured to:
receive, from said camera, said real scene;
graphically overlay said overall image onto said received real scene to determine an augmented reality overall image; and
send said augmented reality overall image to said image display,
v) said image display being configured to:
display said augmented reality overall image.

23. The system according to claim 21, further comprising:
augmented and virtual reality glasses, comprising said camera, coupled to said display.

24. The system according to claim 21, wherein said first position sensors are provided on said ultrasound instrument.

25. The system according to claim 21, wherein said ultrasound instrument comprises an ultrasound probe.

26. The system according to claim 21, further comprising second position sensors provided in fixed positions relative to said ultrasound instrument, wherein said camera is configured to pick up second position data of said ultrasound instrument through said second position sensors.

27. The system according to claim 21, wherein said third position sensors are provided on said medical instrument.

28. The system according to claim 21, wherein said medical instrument comprises an interventional needle.

29. The system according to claim 21, wherein said virtualized image comprises:
a first part of said medical instrument that is directly visible and directly acquirable by said camera;
a second part of the medical instrument that is not directly visible and not directly acquirable by said camera.

30. The system according to claim 26, wherein a positional reference system is defined by said second position sensors and by respective second position data.

31. The system according to claim 30, wherein said georeferenced ultrasound image and said virtualized and georeferenced image of the medical instrument are georeferenced relative to said positional reference system.

32. The system according to claim 21, wherein said second image plane corresponds to a plane in which said virtualized and georeferenced image lies, thus being identified in terms of position in space.

33. The system according to claim 32, wherein said virtualized and georeferenced image is identified in terms of position in space by said virtualized image and said third position data.

34. The system according to claim 33, wherein said virtualized image is graphically constructed on the basis of the position data detected by the first position sensors and a predefined conformation of the medical instrument.

35. The system according to claim 21, wherein said georeferenced ultrasound image is defined in terms of spatial coordinates by said ultrasound image and said first position data.

36. The system according to claim 33, wherein said overlay between said virtualized and georeferenced image of the medical instrument and said georeferenced ultrasound image determines said intersection between said second image plane and said ultrasound beam plane, thus defining said intersection in terms of spatial coordinates.

37. An ultrasound image processing method comprising:
i) providing an ultrasound instrument, configured to contact a patient's body, coupled to first position sensors;
ii) providing a medical instrument, configured for insertion into said patient's body, coupled to third position sensors;
iii) providing a camera in data connection with said instruments and configured to capture a real scene;
iv) providing an image processing device configured to process images originating from said camera and/or from said ultrasound and medical instruments;
v) through said ultrasound instrument:
performing an ultrasound scan by emitting an ultrasound beam and determining an ultrasound image, said ultrasound image lying along an ultrasound beam plane, and
transmitting said ultrasound image to said image processing device;
vi) through said camera:
picking up first position data of said ultrasound instrument through said first position sensors,
transmitting said first position data to said image processing device for a corresponding processing,
acquiring an image of said medical instrument,
picking up third position data of said medical instrument through said third position sensors, and
transmitting said image of said medical instrument and said third position data to said image processing device (50), for a corresponding processing;
vii) through said image processing device:
processing said ultrasound image and said first position data to determine a georeferenced ultrasound image,
graphically positioning said georeferenced ultrasound image along said ultrasound projection beam coming out of said ultrasound instrument along said ultrasound beam plane,
processing said image of said medical instrument and said third position data to determine a virtualized image of said medical instrument,
processing said virtualized image of said medical instrument and said third position data to determine a virtualized and georeferenced image of said medical instrument, said virtualized and georeferenced image lying along a second image plane, and
overlaying said virtualized and georeferenced image of the medical instrument onto said georeferenced ultrasound image so that said second image plane intersects said ultrasound beam plane, the overlaying determining an overall image in which reciprocal positioning of the entire medical instrument relative to said georeferenced ultrasound image is visible.

38. The method according to claim 37, further comprising:
viii) through said image processing device:
receiving, from said camera, said real scene;
graphically overlaying said overall image onto said received real scene to determine an augmented reality overall image;
sending said augmented reality overall image to said image display; and
ix) through said image display:
displaying said augmented reality overall image.

39. The method according to claim 37, further comprising:
providing augmented and virtual reality glasses, comprising said camera, coupled to said display.

40. The method according to claim 37, further comprising:
providing said first position sensors on said ultrasound instrument, and
providing said third position sensors on said medical instrument.
